(19) United States
(12) Patent Application Publication
(10) Pub. No.: US 2020/0297621 A1
(43) Pub. Date: Sep. 24, 2020

(54) Topical Compositions for Neuropathic Pain

(71) Applicant: GOOD PHARMACEUTICAL DEVELOPMENT COMPANY, LLC, Lynbrook, NY (US)

(72) Inventor: Larry I. Good, Lynbrook, NY (US)

(21) Appl. No.: 16/089,434

(22) PCT Filed: Apr. 3, 2017
(86) PCT No.: PCT/US2017/025731

§ 371 Date: Sep. 28, 2018

Related U.S. Application Data
(60) Provisional application No. 62/317,220, filed on Apr. 1, 2016.
(60) Provisional application No. 62/452,117, filed on Jan. 30, 2017.

(51) Int. Cl. A61K 9/00; A61K 31/05; A61K 31/196; A61K 31/13; A61K 47/10; A61K 47/14; A61K 9/06

(52) U.S. Cl. CPC A61K 9/0014; A61K 31/05; A61K 31/196; A61K 9/06; A61K 47/10; A61K 47/14; A61K 31/13

(57) ABSTRACT

Topical compositions are provided containing one or more agents to treat and/or prevent pain. The compositions of the invention can be topically administered to a subject to treat pain, for example, neuropathic pain.

### CROSS REFERENCE TO RELATED APPLICATIONS

This application claims the benefit of U.S. provisional patent applications 62/452,117 filed Jan. 30, 2017 and 62/317,220 filed Apr. 1, 2016, the contents of which are hereby incorporated by reference in their entirety.

### FIELD OF THE INVENTION

The present invention is directed to topical compositions for the treatment of pain, such as neuropathic pain.

### BACKGROUND OF THE INVENTION

Chronic neuropathic pain of multiple etiologies affects more than seven million individuals in the United States and Europe. It is commonly associated with disorders including diabetic neuropathy, spinal cord trauma, post-herpetic neuralgia, Crohn's Disease, pelvic and perineal infection, vulvadynia, and complex regional pain syndrome (causalgia and reflex sympathetic dystrophy).

Currently available treatments are inadequate because of lack of efficacy, adverse effects and the potential for narcotic abuse and addiction. Systemic alternatives that are currently available include orally administrated short and long acting opiates, transcutaneous delivery systems for opiates, gabapentin and pregabalin, as well as off-label use of anti-seizure medications. Topical therapies include anti-inflammatory creams (NSAIDs), capsaicin, and topical lidocaine.

There remains a need for novel therapies for the treatment of neuropathic pain.

### SUMMARY OF THE INVENTION

The present invention is directed to topical compositions comprising one or more agents to treat and/or prevent pain. The compositions of the invention can be topically administered to a subject to treat pain, for example, neuropathic pain.

In one aspect, the present invention is a topical composition comprising (i) about 5% gabapentin; (ii) about 5% ketamine; and (iii) a pharmaceutically acceptable carrier.

In exemplary embodiments, the topical composition is a cream or ointment.

In exemplary embodiments, the topical composition comprises (i) about 5% gabapentin; (ii) about 5% ketamine; (iii) at least one emollient selected from isopropyl myristate, cetyl alcohol, stearyl alcohol or combinations thereof; (iv) glycerine; (v) butylated hydroxytoluen; (vi) at least one emulsifier selected from polyoxyl stearyl ether, PEG-21 stearyl ether and combinations thereof; (vii) at least one preservative selected from propylene glycol, methylparaben and combinations thereof; and (viii) water.

In a second aspect, the present invention is a method of treating pain in a subject in need thereof, comprising topically administering to the subject a composition comprising (i) about 5% gabapentin; (ii) about 5% ketamine; and (iii) a pharmaceutically acceptable carrier, thereby treating pain in the subject.

In exemplary embodiments, the method comprises topically administering a composition comprising (i) about 5% gabapentin; (ii) about 5% ketamine; (iii) at least one emollient selected from isopropyl myristate, cetyl alcohol, stearyl alcohol or combinations thereof; (iv) glycerine; (v) butylated hydroxytoluen; (vi) at least one emulsifier selected from polyoxyl stearyl ether, PEG-21 stearyl ether and combinations thereof; (vii) at least one preservative selected from propylene glycol, methylparaben and combinations thereof; and (viii) water.

In exemplary embodiments, the pain is chronic pain, and more particularly, neuropathic pain. In one embodiment, the neuropathic pain has an etiology selected from post herpetic neuralgia, idiopathic peripheral neuropathy, diabetic neuropathy, chronic radiculopathy, drug or chemotherapy induced neuropathy or chronic regional pain syndrome.

In exemplary embodiments, the topical composition is administered to the site of, or adjacent to, the painful area.

In exemplary embodiments, the total daily dose is between about 1 and about 5 grams of the topical composition. In a particular embodiment, the total daily dose is about 1, about 2, about 3, about 4 or about 5 grams of the topical composition.

In exemplary embodiments, the topical composition is administered once a day or more frequently, such as two, three or four times a day.

In a particular embodiment, the dose is about 1 gram every 6 hours.

In exemplary embodiments, the method produces pain relief without sedation, central nervous adverse effects, addition or combinations thereof.

In a third aspect, the present invention is a method of inducing local anesthesia in a subject in need thereof, comprising topically administering to a subject a composition comprising (i) about 5% gabapentin; (ii) about 5% ketamine; and (iii) a pharmaceutically acceptable carrier, thereby inducing local anesthesia.

In a fourth aspect, the present invention is a topical composition comprising (i) about 5% to about 20% cannabidiol (CBD); and (ii) a pharmaceutically acceptable carrier.

In exemplary embodiments, the topical composition is a cream or ointment.

In exemplary embodiments, the topical composition comprises (i) about 5% to about 20% cannabidiol; and (ii) at least one emollient selected from isopropyl myristate, cetyl alcohol, stearyl alcohol or combinations thereof; (iii) glycerine; (iv) butylated hydroxytoluen; v) at least one emulsifier selected from polyoxyl stearyl ether, PEG-21 stearyl ether and combinations thereof, (vi) at least one preservative selected from propylene glycol, methylparaben and combinations thereof; and (viii) water.

In a fifth aspect, the present invention is a method of treating pain in a subject in need thereof, comprising topically administering to the subject a composition comprising (i) about 5% to about 20% cannabidiol; and (ii) a pharmaceutically acceptable carrier, thereby treating pain in the subject.

In exemplary embodiments, the method comprises topically administering a composition comprising (i) about 5% to about 20% cannabidiol; (ii) at least one emollient selected from isopropyl myristate, cetyl alcohol, stearyl alcohol or combinations thereof; (iii) glycerine; (iv) butylated hydroxytoluen; (v) at least one emulsifier selected from polyoxyl stearyl ether, PEG-21 stearyl ether and combinations thereof; (vi) at least one preservative selected from propylene glycol, methylparaben and combinations thereof; and (vii) water.

In exemplary embodiments, the pain is chronic pain, and more particularly, neuropathic pain. In one embodiment, the neuropathic pain has an etiology selected from post herpetic neuralgia, idiopathic peripheral neuropathy, diabetic neuropathy, chronic radiculopathy, drug or chemotherapy induced neuropathy or chronic regional pain syndrome.

In exemplary embodiments, the topical composition is administered to the site of, or adjacent to, the painful area.

In exemplary embodiments, the total daily dose of cannabidiol is about 10 grams or less but greater than zero. In a particular embodiment, the total daily dose of cannabidiol is between about 8 and about 10 grams, between about 6 and about 8 grams, between about 4 and about 6 grams, between about 2 and about 4 grams.

In exemplary embodiments, the topical composition is administered once a day or more frequently, such as two, three or four times a day.

In exemplary embodiments, the method produces pain relief without sedation, central nervous adverse effects, addition or combinations thereof.

In a third aspect, the present invention is a method of inducing local anesthesia in a subject in need thereof, comprising topically administering to a subject a composition comprising (i) about 5% and about 20% cannabidiol; and (ii) a pharmaceutically acceptable carrier, thereby inducing local anesthesia.

### DETAILED DESCRIPTION

The present invention relates to (topical pharmaceutical formulations of gabapentin and ketamine as well as to topical formulations of cannabidiol, both for the treatment of pain (e.g., neuropathic pain). Advantageously, the compositions of the present invention produce effective pain relief without the side effects of conventional pain therapies.

### Definitions

The term “cannabinoid” as used herein refers to a chemical compound (such as cannabinol, THC or cannabidiol) that is found in the plant species Cannabis saliva (marijuana), and metabolites and synthetic analogues thereof that may or may not have psychoactive properties. Cannabinoids include (without limitation) compounds (such as THC) that have high affinity for the cannabinoid receptor (for example Kᵢ<250 nM), and compounds that do not have significant affinity for the cannabinoid receptor (such as cannabidiol, CBD). Cannabinoids also include compounds that have a characteristic dibenzopyran ring structure (of the type seen in THC) and cannabinoids which do not possess a pyran ring (such as cannabidiol). Hence a partial list of cannabinoids includes THC, CBD, dimethyl heptylpentyl cannabidiol (DMHP-CBD), 6,12-dihydro-6-hydroxy-cannabidiol (described in U.S. Pat. No. 5,227,537, incorporated by reference); (3S,4R)-7-hydroxy-Δ⁶-tetrahydrocannabinol homologs and derivatives described in U.S. Pat. No. 4,876,276, incorporated by reference; (+)-4-[4-DMH-2,6-diacetoxy-phenyl]-2-carboxy-6,6-dimethylbicyclo[3.1.1]hept-2-en, and other 4-phenylpinene derivatives disclosed in U.S. Pat. No. 5,434,295, which is incorporated by reference; and cannabidiol (−)(CBD) analogs such as (−)CBD-monomethylethther, (−)CBD dimethyl ether: (−)CBD diacetate; (−)3′-acetyl-CBD monoacetate; and ±AF11, all of which are disclosed in Consroe et al., J Cln. Pharmacol. 21:428S-436S, 1981, which is also incorporated by reference, Many other cannabinoids are similarly disclosed in Agurell et al., Pharmacol. Rev. 38:31-43, 1986, which is also incorporated by reference.

The term “carrier” as used herein refers to organic or inorganic ingredients, natural or synthetic, with which an active ingredient is combined to facilitate application of a composition. In the present context, the terms “carrier” and “vehicle” are interchangeably used. The term “carrier” includes, but is not limited to, water, acetone, alone or in combination with materials such as silicone fluids.

The term “cream” as used herein refers to viscous liquids or semisolid emulsions, either oil-in-water or water-in-oil.

The terms “comprising,” “including,” “containing” and the like as used herein are inclusive or open-ended terms that do not exclude additional, un-recited elements or method steps.

To the extent used herein, the phrase “consisting of” and grammatical equivalents thereof exclude any element, step, or ingredient not specified in the claim.

To the extent used herein, the phrase “consisting essentially of” limits the scope of a claim to the specified materials or steps and those that do not materially affect the basic and novel characteristic or characteristics of the claimed disclosure.

The term “dosage unit” as used herein refers to a single unit of the dosage form that is to be administered to the patient. The dosage unit will be typically formulated to include an amount of drug sufficient to achieve a therapeutic effect with a single administration. More than one dosage unit may be necessary to administer sufficient drug to achieve a therapeutic effect where the amount of drug causes physical constraints on the size of the dosage form.

The term “emollient” as used herein refers to an agent that softens and smooth's the skin. They are used to correct dryness and scaling of the skin. Emollients are also occlusive agents, i.e., substances that provide a layer of protection that helps prevent moisture (water) loss from the skin. An emollient may be, for example, selected from the group consisting of fats, oils, fatty alcohols, fatty acids, fatty acid ethers and fatty acid esters and mixtures thereof. Several emollients may be present in a single composition, selected for their individual properties and blended to achieve a desired result.

The term “humectant” as used herein refers to a substance that bonds to water molecules to increase the water content in the skin itself. Many humectants also have emollient properties, although not all emollients are humectants. Several humectants may be present in a single composition, selected for their individual properties and blended to achieve a desirable result.

The term “foam” as used herein refers to an emulsion in combination with a gaseous propellant.

The term “gel” as used herein refers to a semisolid system containing dispersions of small or large molecules in a liquid vehicle that is rendered semisolid by the action of a thickening agent or polymeric material dissolved or suspended in the liquid vehicle.

The term “lessening” as used herein refers in context herein to the percentage changes, e.g., by about 5%, about 10%, about 20%, about 30%, about 40%, about 50%, about 60%, about 70%, about 80%, about 90%, about 95%, about 99%, about 125%, etc., or even more, e.g., 2 or 4 fold, or even more.

The term “local administration” as used herein encompasses topical administration, as well as administration to spatially restricted portions of the body, including portions of the skin, muscle, eyes, and other tissues and organs, and combinations of these.

The term “lotion” as used herein refers to preparations to be applied to the skin surface without friction and are typically liquid or semiliquid preparation in which solid particles, including the active agents, are present in a water or alcohol base.

The term “neuropathic pain” as used herein refers to any and all types of neuropathic pain regardless of the etiology. Examples of neuropathic pain include, but are not limited to, thermal or mechanical hyperalgesia, thermal or mechanical allodynia, painful diabetic neuropathy, post-herpetic neuralgia, phantom limb pain, sciatica, chemotherapy-induced neuropathy, HIV-AIDS-associated neuropathy, nerve entrapment pain, and the like.

The term “ointment” as used herein refers to a semisolid preparation containing an ointment base and optionally one or more active agents.

The term “paste” as used herein refers to semisolid dosage forms in which the active agent is suspended in a suitable base. Depending on the nature of the base, pastes are divided between fatty pastes or those made from a single-phase aqueous gels.

The term “pharmacologically active agent” as used herein refers to a chemical material or compound that, when administered to a subject (e.g., a human) induces a desired pharmacologic effect, such as a reduction in pain.

The term “pharmaceutically acceptable” as used herein, such as in the recitation of a “pharmaceutically acceptable carrier,” or a “pharmaceutically acceptable acid addition salt,” means that a material is not biologically or otherwise undesirable, i.e., the material may be incorporated into a pharmaceutical composition administered to a patient without causing any undesirable biological effects or interacting in a deleterious manner with any of the other components of the composition in which it is contained. The term “pharmacologically active” (or simply “active”) as in a “pharmacologically active” derivative, refers to a derivative having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well. When the term, “pharmaceutically acceptable” is used to refer to an excipient, it implies that the excipient has met the required standards of toxicological and manufacturing testing or that it is on the Inactive Ingredient Guide prepared by the FDA, or comparable agency.

The term “preservative” as used herein refers to a natural or synthetic chemical that is added to products to prevent decomposition by microbial growth or by undesirable chemical changes

The terms “skin permeation enhancer” or “skin penetration enhancer” or “penetration enhancer” as used herein refers to a component used to enhance the penetration rate of drugs through the skin or mucous membrane, such as by temporarily diminishing the impermeability of the skin or membrane. Permeation enhancers have also been called “accelerants” and “absorption promoters.”

The terms “patient,” “subject,” or “host” as used herein may mean either a human or non-human animal, such as primates, mammals, and vertebrates. Preferably, a “subject” is a human.

The term “therapeutically effective amount” as used herein means a sufficient amount of a compound or composition to provide a therapeutic benefit in the treatment or management of a disease or condition, or to delay or minimize one or more symptoms associated with the disease or condition. An “therapeutically effective amount” can encompass an amount that improves overall therapy, reduces or avoids symptoms or causes of a disease or condition, or enhances the therapeutic efficacy of another therapeutic agent. When applied to an individual active ingredient, administered alone, the term refers to that ingredient alone. When applied to a combination, the term refers to combined amounts of the active ingredients that result in the therapeutic effect, whether administered in combination, serially, or simultaneously. The amount and timing of compositions administered will, of course, be dependent on the subject being treated, on the severity of the affliction, on the manner of administration and on the judgment of the prescribing physician. Thus, because of patient-to-patient variability, the dosages given above are a guideline and the physician may titrate doses of the drug to achieve the treatment that the physician considers appropriate for the patient. In considering the degree of treatment desired, the physician must balance a variety of factors such as age of the patient, presence of preexisting disease, as well as presence of other diseases.

The term “topical administration” as used herein means delivery of a pharmacologically active agent to the skin or mucosa, including the mucosa of the mouth, nasal and sinus cavities, eyes, gastrointestinal tract, bladder, urethra, and vagina.

The term “total daily dose” as used herein refers to the total amount of compound or composition administered to the patient in one 24 hour period, regardless of whether the protocol calls for a once-daily, twice-daily, or thrice-daily administration of the compound or composition. Thus, the total amount of compound or composition is summed for a given 24 hour period to determine how much total compound or composition the subject is to be administered in a given day.

Composition

The topical compositions of the present invention comprise a therapeutically effective amount of one or more therapeutic and/or preventive agents for pain in a pharmaceutically acceptable carrier. The topical composition may be prepared in a variety of physical forms. The primary product forms are creams, lotions; gels/serums, and aqueous liquids. The principal differences between these forms are their physical appearance and viscosity (or thickness), which are governed primarily by the presence and amount of emulsifiers and viscosity adjusters.

I. Gabapentin and Ketamine

In one aspect, the present invention provides a topical composition comprising (i) gabapentin; (ii) ketamine; and (iii) a pharmaceutically acceptable carrier. As used herein, the terms “gabapentin” and “ketamine” include the agent itself, as well as pharmaceutically acceptable salt forms, hydrates or solvates that retain the biological effectiveness and properties of the agent.

Gabapentin (1-(aminomethyl)cyclohexane acetic acid) is a 3-substituted gamma-aminobutyric acid (“GABA”) analog approved in the United States as NEURONTIN. It has the molecular formula C₉H₁₇NO₂ and a molecular weight of 171.24. It is available in capsule (100 mg, 300 mg, 400 mg), tablet (600 mg, 800 mg) and oral solution (25 mg/5 mL). The structural formula of gabapentin is:

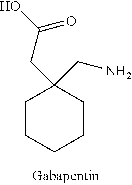 [SMILES: NCC1(CC(=O)O)CCCCC1]

Ketamine (racemic mixture of the corresponding S- and R-enantiomers) is an NMDA receptor antagonist, with a wide range of effects in humans, including analgesia, anesthesia, hallucinations, dissociative effects, elevated blood pressure and bronchodilation. Ketamine is primarily used for the induction and maintenance of general anesthesia.

Ketamine hydrochloride is a non-barbiturate anesthetic chemically designated as (±) 2-(o-chlorophenyl)-2-(methylamino)-cyclohexanone. It is formulated as a slightly acid (pH 3.5 to 0.5) sterile solution for intravenous or intramuscular injection in concentration containing the equivalent of either 50 mg or 100 mg ketamine base per milliliter and contains no more than 0.1 mg/mL benzethonium chloride added as a preservative. It has the molecular formula C13H16ClNo.HCL, a molecular weight of 27419 and the following structural formula:

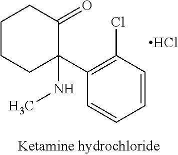 [SMILES: CNC1(c2ccccc2Cl)CCCCC1=O ; Cl]

The therapeutically effective amount of ketamine and/or gabapentin may vary. In exemplary embodiments, the therapeutically effective amount of ketamine is about 0.0001% to about 25% w/w. In another embodiment, the therapeutically effective amount of ketamine is about 20% w/w, about 15% w/w, about 10% w/w, about 5% w/w, or about 1% w/w. In one embodiment, the therapeutic amount of ketamine is about 0.0001% to about 10% w/w, about 0.005% to about 5% w/w, or about 0.01% to about 5% w/w. In still a further embodiment, the therapeutically effective amount of ketamine is about 0.01% w/w, about 0.05% w/w, about 0.10% w/w, about 0.20% w/w, about 0.30% w/w, about 0.40% w/w, about 0.50% w/w, about 0.60% w/w, about 0.70% w/w, about 0.80% w/w, about 0.90% w/w, about 1.0% w/w, about 2.0% w/w, about 3.0% w/w, about 4.0% w/w, about 5.0% w/w, about 6.0% w/w, about 7.0% w/w, about 8.0% w/w, about 9.0% w/w or about 10.0% w/w.

In a preferred embodiment, the therapeutically effective amount of ketamine is about 5.0% w/w. In one embodiment, the therapeutically effective amount of ketamine is about 4.2% w/w, about 4.4% w/w, about 4.6% w/w, about 4.8% w/w, about 5.2% w/w, about 5.4% w/w or about 5.6% w/w.

In exemplary embodiments, the therapeutically effective amount of gabapentin is about 0.0001% to about 25% w/w. In another embodiment, the therapeutically effective amount of gabapentin is less than about 20% w/w, about 15% w/w, about 10% w/w, about 5% w/w, or about 1% w/w. In another embodiment, the therapeutically effective amount of gabapentin is about 0.0001% to about 10% w/w, about 0.005% to about 5% w/w, or about 0.01% to about 5% w/w. In still a further embodiment, the therapeutically effective amount of gabapentin is about 0.01% w/w, about 0.05% w/w, about 0.10% w/w, about 0.20% w/w, about 0.30% w/w, about 0.40% w/w, about 0.50% w/w, about 0.60% w/w, about 0.70% w/w, about 0.80% w/w, about 0.90% w/w, about 1.0% w/w, about 2.0% w/w, about 3.0% w/w, about 4.0% w/w, about 5.0% w/w, about 6.0% w/w, about 7.0% w/w, about 8.0% w/w, about 9.0% w/w or about 10.0% w/w.

In a preferred embodiment, the therapeutically effective amount of gabapentin is about 5.0% w/w. In one embodiment, the therapeutically effective amount of gabapentin is about 0.2% w/w, about 4.4% w/w, about 4.6% w/w, about 4.8% w/w, about 5.2% w/w, about 5.4% w/w or about 5.6% w/w.

In exemplary embodiments, the therapeutically effective amount of ketamine is between about 1% and about 10% w/w and the amount of gabapentin is between about 1% and about 10% w/w.

In exemplary embodiments, the therapeutically effective amount of ketamine is about 5% w/w and the amount of gabapentin is between about 1% and about 10% w/w, about 3% w/w and about 5% w/w, or about 5% w/w.

In exemplary embodiments, the therapeutically effective amount of gabapentin is about 5% w/w and the amount of ketamine is between about 1% and about 10% w/w, about 3% w/w and about 5% w/w, or about 5% w/w.

The compositions of this invention can also be administered topically to a subject, e.g., by the direct lying on or spreading of the composition on the epidermal or epithelial tissue of the subject, or transdermally via a “patch”. The pharmaceutically acceptable carrier may be in the form of an ointment, cream, lotion, gel, paste, a solid “stick”, foam, solution, balms, sprays, suspensions, ointments, films or the like, that can be applied to the skin by hand, for example, by rubbing or spraying.

In exemplary embodiments, the carrier is in the form of a cream. In exemplary embodiment, the carrier is in the form of an ointment.

The compositions of the invention can further comprise one or more additional pharmaceutically acceptable excipients, such as one or more thickening agents, medicinal agents or pharmaceuticals, bioadhesive polymers, inert carriers, lipid absorbents, viscosity stabilizers, chelating agents, buffers, anti-fading agents, stabilizers, moisture absorbents, fragrances, colorants, film-forming materials or re-fatting agents. Various enhancements may be made to the composition to improve order, scent or aroma of the topical composition. Preservatives can be used to prevent the growth of fungi and microorganisms.

In exemplary embodiments, the present invention provides a topical composition comprising (i) a therapeutically effective amount of ketamine; (ii) a therapeutically effective amount of gabapentin; and (iii) a pharmaceutically acceptable carrier. In exemplary embodiments, the topical composition is in the form of a cream or ointment.

In exemplary embodiments, the present invention provides a topical composition comprising (i) between about 1% and about 10% w/w ketamine; (ii) between about 1% and about 10% w/w gabapentin; and (iii) a pharmaceutically acceptable carrier. In exemplary embodiments, the topical composition is in the form of a cream or ointment.

In exemplary embodiments, the present invention provides a topical composition comprising (i) about 5% w/w ketamine; (ii) about 5% w/w gabapentin; and (iii) a pharmaceutically acceptable carrier. In exemplary embodiments, the topical composition is in the form of a cream or ointment.

In exemplary embodiments, the present invention provides a topical composition that comprises (i) about 5% w/w ketamine; (ii) about 5% w/w gabapentin; and (iii) at least one emollient. In certain embodiments, the at least one emollient is selected from a fatty alcohol, a fatty ester or combinations thereof. Representative, non-limiting examples of fatty alcohols include lauryl alcohol, cetyl alcohol, stearyl alcohol, jojoba alcohol and oleyl alcohol. In a preferred embodiment, representative, non-limiting examples of fatty esters include palmitate, isopropyl myristate and glyceryl stearate.

In certain embodiments, the at least one emollient is present in the composition in an amount from about 1.0% w/w to about 30% w/w, about 5% w/w to about 25% w/w, about 10% w/w to about 15% w/w. In a particular embodiment, the at least one emollient is present in the composition in an amount of about 1.0% w/w, about 5.0% w/w, about 10% w/w, about 15% w/w, about 20% w/w, or about 25% w/w.

In a particular embodiment, the present invention provides a topical composition comprising (i) about 5% w/w ketamine; (ii) about 5% w/w gabapentin; and (iii) at least one emollient selected from isopropyl myristate, cetyl alcohol, stearyl alcohol and combinations thereof. Optionally, the composition further comprises one or more additional components selected from a humectant, an antioxidant, an emulsifier, a preservative and combinations thereof.

In a particular embodiment, the present invention provides a topical composition comprising (i) about 5% w/w ketamine; (ii) about 5% w/w gabapentin; and (iii) at least two emollients.

In a particular embodiment, the present invention provides a topical composition comprising (i) about 5% w/w ketamine; (ii) about 5% w/w gabapentin; and (iii) at least three emollients.

In a particular embodiment, the present invention provides a topical composition comprising (i) about 5% w/w ketamine; (ii) about 5% w/w gabapentin; and (iii) at least three emollients, wherein the at least three emollients include isopropyl myristate, cetyl alcohol, and stearyl alcohol.

In another particular embodiment, the present invention provides a topical composition comprising (i) about 5% w/w ketamine; (ii) about 5% w/w gabapentin; and (iii) at least three emollients, wherein the at least three emollients include isopropyl myristate, cetyl alcohol, and stearyl alcohol, wherein the isopropyl myristate is present in the composition in an amount from about 5% to about 15% w/w, the cetyl alcohol is present in the composition in an amount from about 1% to about 10% w/w and the stearyl alcohol is present in the composition in an amount from about 1% to about 10% w/w.

In an exemplary embodiment, the present invention provides a topical composition comprising (i) about 5% w/w ketamine; (ii) about 5% w/w gabapentin; and (iii) at least three emollients, wherein the at least three emollients include isopropyl myristate, cetyl alcohol, and stearyl alcohol, wherein the isopropyl myristate is present in the composition in an amount of about 10% w/w, the cetyl alcohol is present in the composition in an amount of about 5% w/w and the stearyl alcohol is present in the composition in an amount of about 5% w/w.

In exemplary embodiments, the present invention provides a topical composition comprising (i) about 5% w/w ketamine; (ii) about 5% w/w gabapentin; and (iii) at least one humectant. Representative, non-limiting examples of humectants include glycerin, sorbitol, propylene glycol, macrogols, maltodextrin, and wheat extracts.

In certain embodiments, the at least one humectant is present in the composition in an amount from about 0.1% w/w to about 10% w/w, about 1% w/w to about 5% w/w, or about 2.5% w/w. In a particular embodiment, the at least one emollient is present in the composition in an amount of about 0.1% w/w, about 1.0% w/w, about 1.5% w/w, about 2.0% w/w, about 2.5% w/w, about 3.0% w/w or about 3.5% w/w,

In a particular embodiment, the present invention provides a topical composition comprising (i) about 5% w/w ketamine; (ii) about 5% w/w gabapentin; and (iii) glycerin. Optionally, the composition further comprises one or more additional components selected from an emollient, an antioxidant, an emulsifier, a preservative and combinations thereof.

In a particular embodiment, the present invention provides a topical composition comprising (i) about 5% w/w ketamine; (ii) about 5% w/w gabapentin; and (iii) glycerin, wherein glycerin is present in an amount from about 0.1% w/w to about 10% w/w, or more particularly, from about 1.0% w/w to about 5% w/w, or even more particularly, about 2.5% w/w.

In exemplary embodiments, the present invention provides a topical composition comprising (i) about 5% w/w ketamine; (ii) about 5% w/w gabapentin; and (iii) at least one antioxidant. Representative, non-limiting examples of antioxidants include synthetic antioxidants such as BHT (butylalted hydroxytoluene), BHA (butylated hydroxyanisole) and TBHQ (tertiary butyl hydroquinone). Other anti-oxidants suitable for use include acetyl cysteine, ascorbic acid polypeptide, ascorbyl dipalmitate, ascorbyl methylsilanol pectinate, ascorbyl palmitate, ascorbyl stearate, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

In certain embodiments, the at least one antioxidant is present in the composition in an amount from about 0.01% w/w to about 5% w/w, about 0.10% w/w to about 1% w/w, or about 0.10% to about 0.50% w/w. In a particular embodiment, the at least one antioxidant is present in the composition in an amount of about 0.05% w/w, about 0.10% w/w, about 0.20% w/w, about 0.30% w/w, about 0.40% w/w, or about 0.5% w/w.

In a particular embodiment, the present invention provides a topical composition comprising (i) about 5% w/w ketamine; (ii) about 5% w/w gabapentin; and (iii) butylalted hydroxytoluene. Optionally, the composition further comprises one or more additional components selected from an emollient, a humectant, an emulsifier, a preservative and combinations thereof.

In a particular embodiment, the present invention provides a topical composition comprising (i) about 5% w/w ketamine; (ii) about 5% w/w gabapentin; and (iii) butylalted hydroxytoluene, wherein the butylalted hydroxytoluene is present in an amount from 0.01% w/w to about 5% w/w, more particularly about 0.10% w/w to about 1% w/w, or even more particularly, about 0.20% w/w.

In exemplary embodiments, the present invention provides a topical composition comprising (i) about 5% w/w ketamine; (ii) about 5% w/w gabapentin; and (iii) at least one emulsifier. Representative, non-limiting examples of emulsifiers include glycol esters, fatty acids, fatty alcohols, fatty acid glycol esters, fatty esters, fatty ethers, esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, derivatives thereof, and mixtures thereof.

In certain embodiments, the at least one emulsifier is present in an amount from about 0.01% w/w to about 5% w/w, about 0.10% w/w to about 1% w/w, or about 0.10% to about 0.50% w/w. In a particular embodiment, the at least one antioxidant is present in the composition in an amount of about 0.05% w/w, about 0.10% w/w, about 0.20% w/w, about 0.30% w/w, about 0.40% w/w, about 0.5% w/w, about 0.6% w/w, about 0.7% w/w, about 0.8% w/w, about 1.0% w/w, about 1.5% w/w, or about 2.0% w/w.

In a particular embodiment, the present invention provides a topical composition comprising (i) about 5% w/w ketamine; (ii) about 5% w/w gabapentin; and (iii) polyoxyl stearyl ether.

In a particular embodiment, the present invention provides a topical composition comprising (i) about 5% w/w ketamine; (ii) about 5% w/w gabapentin; and (iii) polyoxyl stearyl ether, wherein the polyoxyl stearyl ether is present in an amount from about 0.10% w/w to about 1.0% w/w, or more particularly about 0.40% w/w to about 0.50% w/w, or even more particularly, about 0.47% w/w.

In a particular embodiment, the present invention provides a topical composition comprising (i) about 5% w/w ketamine; (ii) about 5% w/w gabapentin; and (iii) PEG 21 stearyl ether.

In a particular embodiment, the present invention provides a topical composition comprising (i) about 5% w/w ketamine; (ii) about 5% w/w gabapentin; and (iii) PEG 21 stearyl ether, wherein the PEG 21 stearyl ether is present in an amount from about to about 0.10% w/w to about 10% w/w, or more particularly about 1.0% w/w to about 5% w/w, or even more particularly, about 2.0%.

In a particular embodiment, the present invention provides a topical composition comprising (i) about 5% w/w ketamine; (ii) about 5% w/w gabapentin; and at least two emulsifiers.

In a particular embodiment, the present invention provides a topical composition comprising (i) about 5% w/w ketamine; (ii) about 5% w/w gabapentin; and at least two emulsifiers, wherein the at least two emulsifiers are present in an amount from 0.10% w/w to about 10% w/w, or more particularly about 1.0% w/w to about 5% w/w, or even more particularly, about 2.0% w/w to about 3.0% w/w.

In one embodiment, the present invention provides a topical composition comprising (i) about 5% w/w ketamine; (ii) about 5% w/w gabapentin; and (iii) at least two emulsifiers selected wherein the at least two emulsifiers comprise polyoxyl stearyl ether and PEG 21 stearyl ether. Optionally, the composition further comprises one or more additional components selected from an emollient, a humectant, an antioxidant, a preservative and combinations thereof.

In exemplary embodiments, the present invention provides a topical composition comprising (i) about 5% w/w ketamine; (ii) about 5% w/w gabapentin; and (iii) at least one preservative to inhibit the growth of pathogens over an extended period. Representative, non-limiting examples of suitable preservatives include sorbic acid, benzoic acid, methyl-paraben, propyl-paraben, methyl chloroisothiazolinone, metholisothiazolinone, diazolidinyl urea, chlorobutanol, triclosan, benzethonium chloride, p-hydroxybenzoate, chlorhexidine, digluconate, hexadecyltrimethyl ammonium bromide, alcohols, benzalkonium chloride, boric acid, bronopol, butylparaben, butylene calcium acetate, calcium chloride, calcium lactate, carbon dioxide, cationic, and bentonite, cetrimide, cetylpyridinium chloride, chlorhexidine, chlorobutanol, chlorocresol, chloroxylenol, citric acid monohydrate, cresol, dimethyl ether, ethylparaben, glycerin, hexetidine, imidurea, isopropyl alcohol, lactic acid, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium sorbate, propionic acid, propyl gallate, propylene glycol, sodium acetate, sodium benzoate, sodium borate, sodium lactate, sodium sulfite, sodium propionate, sodium metabisulfite, xylitol, sulphur dioxide, carbon dioxide, and any combination thereof.

Preservatives, when used, are typically present in an amount from about 0.01 to 6 weight percent. In exemplary embodiments, the at least one preservative is present in an amount from about 0.01% w/w to about 5% w/w, about 0.10% w/w to about 5% w/w, about 0.50% w/w to about 5% w/w, or about 1.0% w/w to about 3.0% w/w.

In a particular embodiment, the present invention provides a topical composition comprising (i) about 5% w/w ketamine; (ii) about 5% w/w gabapentin 5% w/w gabapentin; and (iii) at least one preservative selected from propylene glycol, methylparaben and combinations thereof.

In a particular embodiment, the present invention provides a topical composition comprising (i) about 5% w/w ketamine; (ii) about 5% w/w gabapentin 5% w/w gabapentin; and (iii) at least one preservative selected from propylene glycol, methylparaben and combinations thereof, wherein the at least one preservative is present in an amount from about 1.0% w/w to about 5% w/w, or more particularly about 3% w/w to about 4% w/w.

Water may be present in the composition present invention. In exemplary embodiments, water is present in an amount of about 40% to about 99% by weight of the composition, or any range and/or individual value therein, such as, but not limited to, about 55% to about 95% or about 60% to about 70% by weight of the first composition. In certain embodiments, water is present in a first composition of the present invention in an amount of about 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, or 79% by weight of the first composition or any range and/or individual value therein.

The compositions of the present invention optionally include one or more the composition may also include one or more penetration enhancers. Representative, non-limiting penetration enhancers include C₅-C₄₄ fatty alcohols, preferably C₅-C₂₀ fatty alcohols. These fatty alcohols belong to the group of long chain saturated fatty alcohols, unsaturated chain fatty alcohol, branched chain alcohol or combinations thereof.

The compositions of the present invention optionally include one or more stiffening agents. Suitable fragrances and colors may be used in the compositions.

In one embodiment, the present invention is the topical composition disclosed in Example 1.

The composition may be presented in unit dose forms containing a predetermined amount of active ingredient per unit dose. In exemplary embodiments, the unit dosage form is one gram of cream.

II. Cannabidiol

In a second aspect, the present invention provides a topical composition comprising (i) cannabidiol; and (ii) a pharmaceutically acceptable carrier. As used herein, the terms “cannabidiol” include the agent itself, as well as pharmaceutically acceptable salt forms, hydrates or solvates that retain the biological effectiveness and properties of the agent. Cannabidiol derivatives are also contemplated, including for example, cannabidiol dimethyl hephtyl (CBD-DMH) and CBD-DMH-7-oic acid (HU-320).

Cannabidiol (CBD) is one of more than 80 active cannabinoids identified in cannabis. Its formula is C21H30O2 and it has a molecular mass of 314.4636. Unlike the main psychoactive cannabinoid in marijuana, tetrahydrocannabinol (THC), CBD does not produce euphoria or intoxication.

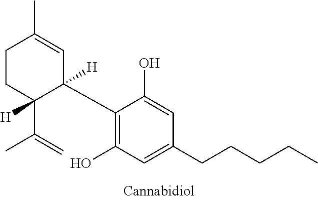 [SMILES: C=C(C)[C@@H]1CCC(C)=C[C@H]1c1c(O)cc(CCCCC)cc1O]

Cannabidiol was formerly regarded as an inactive constituent, however there is emerging evidence that it has pharmacological activity, which is different from that of THC in several respects. CBD constitutes up to 40% of the extracts of the plant. However, CBD concentrations are variable and depend on the growing conditions, the different phenotypes of cannabis and on the part of the plant analyzed.

In a particular embodiment, CBD is derived from a CBD-rich strain of cannabis. In one embodiment, CBD content level that is 4% or more. In another embodiment, the CBD content is between about 5 and about 10%, between about 10 and about 20%, between about 20 and about 25% or about 25% or more. In a particular embodiment, the CBD content is about 5%, about 8%, about 10%, about 12%, about 15%, about 18%, about 20% or about 24% or more.

In another embodiment, the CBD is derived from a strain that is low in THC. In one embodiment, the strains has less than about 10.0%, less than about 5%, less than about 3.0%, less than about 2.0%, less than about 1.0% or less than about 0.5% THC—but in each case greater than 0% THC. In another embodiment, CBD is derived from a strain having 0% THC.

In one embodiment, CBD is derived from a strain having a CBD:THC ratio of between about 20:1 and about 1:1. In a particular embodiment, the CBD:THC ratio is about 20:1, about 15:1, about 10:1, about 3:2 or about 1:1.

In one embodiment CBD is provided as cannabis oil concentrate, and more particularly, a cannabis oil concentrate derived from made from high-CBD, low-THC hemp.

U.S. Pat. No. 2,304,669 discloses a multiple step method for isolating CBD from plant extracts, the process involves the treatment of oil derived from cannabis plants with 3,5-dinitrobenzoylchloride to form cannabidiol bis-3,5-dinitrobenzoate, separating this mixture from the oil and then subjecting this benzoate ester to ammonolysis to produce purified cannabidiol.

U.S. Patent No. US20060167283 describes a method of production of cannabidiol which is said to be inexpensive and yet capable of yielding substantially pure cannabidiol.

The therapeutically effective amount of CBD may vary. In exemplary embodiments, the therapeutically effective amount of CBD is about 0.0001% to about 50% w/w. In another embodiment, the therapeutically effective amount of CBD is about 50% w/w, about 45% w/w, about 40% w/w, about 35% w/w, about 35% w/w, about 20% w/w, about 15% w/w, about 10% w/w, about 5% w/w, or about 1% w/w. In one embodiment, the therapeutic amount of CBD is about 0.0001% to about 10% w/w, about 0.005% to about 5% w/w, or about 0.01% to about 5% w/w. In still a further embodiment, the therapeutically effective amount of CBD is about 0.01% w/w, about 0.05% w/w, about 0.10% w/w, about 0.20% w/w, about 0.30% w/w, about 0.40% w/w, about 0.50% w/w, about 0.60% w/w, about 0.70% w/w, about 0.80% w/w, about 0.90% w/w, about 1.0% w/w, about 2.0% w/w, about 3.0% w/w, about 4.0% w/w, about 5.0% w/w, about 6.0% w/w, about 7.0% w/w, about 8.0% w/w, about 9.0% w/w or about 10.0% w/w.

In a preferred embodiment, the therapeutically effective amount of CBD is about 5.0% to about 20% w/w.

The compositions of this invention can also be administered topically to a subject, e.g., by the direct lying on or spreading of the composition on the epidermal or epithelial tissue of the subject, or transdermally via a “patch”. The pharmaceutically acceptable carrier may be in the form of an ointment, cream, lotion, gel, paste, a solid “stick”, foam, solution, balms, sprays, suspensions, ointments, films or the like, that can be applied to the skin by hand, for example, by rubbing or spraying.

In exemplary embodiments, the carrier is in the form of a cream. In exemplary embodiments, the carrier is in the form of an ointment.

The compositions of the invention can further comprise one or more additional pharmaceutically acceptable excipients, such as one or more thickening agents, medicinal agents or pharmaceuticals, bioadhesive polymers, inert carriers, lipid absorbents, viscosity stabilizers, chelating agents, buffers, anti-fading agents, stabilizers, moisture absorbents, fragrances, colorants, film-forming materials or re-fatting agents. Various enhancements may be made to the composition to improve order, scent or aroma of the topical composition. Preservatives can be used to prevent the growth of fungi and microorganisms.

In exemplary embodiments, the present invention provides a topical composition comprising (i) therapeutically effective amount of CBD; and (iii) a pharmaceutically acceptable carrier. In exemplary embodiments, the topical composition is in the form of a cream or ointment.

In exemplary embodiments, the present invention provides a topical composition comprising (i) between about 5% and about 20% w/w CBD; and (iii) a pharmaceutically acceptable carrier. In exemplary embodiments, the topical composition is in the form of a cream or ointment.

In exemplary embodiments, the present invention provides a topical composition comprising (i) about 5% to about 10% w/w CBD; and (iii) a pharmaceutically acceptable carrier. In exemplary embodiments, the topical composition is in the form of a cream or ointment.

In exemplary embodiments, the present invention provides a topical composition comprising (i) about 10% to about 20% w/w CBD; and (iii) a pharmaceutically acceptable carrier. In exemplary embodiments, the topical composition is in the form of a cream or ointment.

In exemplary embodiments, the present invention provides a topical composition comprising (i) about 5% w/w CBD; and (ii) a pharmaceutically acceptable carrier. In exemplary embodiments, the topical composition is in the form of a cream or ointment.

In exemplary embodiments, the present invention provides a topical composition comprising (i) about 10% w/w CBD; and (ii) a pharmaceutically acceptable carrier. In exemplary embodiments, the topical composition is in the form of a cream or ointment.

In exemplary embodiments, the present invention provides a topical composition comprising (i) about 15% w/w CBD; and (ii) a pharmaceutically acceptable carrier. In exemplary embodiments, the topical composition is in the form of a cream or ointment.

In exemplary embodiments, the present invention provides a topical composition comprising (i) about 20% w/w CBD; and (ii) a pharmaceutically acceptable carrier. In

In one embodiment, the present invention provides a topical composition comprising (i) about 5%, about 8%, about 10%, about 12%, about 15%, about 18%, about 20% or about 25% w/w CBD; and (ii) a pharmaceutically acceptable carrier.

In exemplary embodiments, the topical composition is in the form of a cream or ointment.

In exemplary embodiments, the present invention provides a topical composition that comprises (i) about 5% to about 20% w/w CBD; and (ii) at least one emollient. In certain embodiments, the at least one emollient is selected from a fatty alcohol, a fatty ester or combinations thereof. Representative, non-limiting examples of fatty alcohols include lauryl alcohol, cetyl alcohol, stearyl alcohol, jojoba alcohol and oleyl alcohol. In a preferred embodiment, representative, non-limiting examples of fatty esters include palmitate, isopropyl myristate and glyceryl stearate.

In certain embodiments, the at least one emollient is present in the composition in an amount from about 1.0% w/w to about 30% w/w, about 5% w/w to about 25% w/w, about 10% w/w to about 15% w/w. In a particular embodiment, the at least one emollient is present in the composition in an amount of about 1.0% w/w, about 5.0% w/w, about 10% w/w, about 15% w/w, about 20% w/w, or about 25% w/w.

In a particular embodiment, the present invention provides a topical composition comprising (i) about 5% to about 20% w/w CBD; and (ii) at least one emollient selected from isopropyl myristate, cetyl alcohol, stearyl alcohol and combinations thereof. Optionally, the composition further comprises one or more additional components selected from a humectant, an antioxidant, an emulsifier, a preservative and combinations thereof.

In a particular embodiment, the present invention provides a topical composition comprising (i) about 5% to about 20% w/w CBD and (ii) at least two emollients.

In a particular embodiment, the present invention provides a topical composition comprising (i) about 5% to about 20% w/w CBD; and (ii) at least three emollients.

In a particular embodiment, the present invention provides a topical composition comprising (i) about 5% to about 20% w/w CBD; and (ii) at least three emollients, wherein the at least three emollients include isopropyl myristate, cetyl alcohol, and stearyl alcohol.

In another particular embodiment, the present invention provides a topical composition comprising (i) about 5% to about 20% w/w CBD; and (ii) at least three emollients, wherein the at least three emollients include isopropyl myristate, cetyl alcohol, and stearyl alcohol, wherein the isopropyl myristate is present in the composition in an amount from about 5% to about 15% w/w, the cetyl alcohol is present in the composition in an amount from about 1% to about 10% w/w and the stearyl alcohol is present in the composition in an amount from about 1% to about 10% w/w.

In an exemplary embodiment, the present invention provides a topical composition comprising (i) about 5% to about 20% w/w CBD; and (ii) at least three emollients, wherein the at least three emollients include isopropyl myristate, cetyl alcohol, and stearyl alcohol, wherein the isopropyl myristate is present in the composition in an amount of about 10% w/w, the cetyl alcohol is present in the composition in an amount of about 5% w/w and the stearyl alcohol is present in the composition in an amount of about 5% w/w.

In exemplary embodiments, the present invention provides a topical composition comprising (i) about 5% to about 20% w/w CBD; and (ii) at least one humectant. Representative, non-limiting examples of humectants include glycerin, sorbitol, propylene glycol, macrogols, maltodextrin, and wheat extracts.

In certain embodiments, the at least one humectant is present in the composition in an amount from about 0.1% w/w to about 10% w/w, about 1% w/w to about 5% w/w, or about 2.5% w/w. In a particular embodiment, the at least one emollient is present in the composition in an amount of about 0.1% w/w, about 1.0% w/w, about 1.5% w/w, about 2.0% w/w, about 2.5% w/w, about 3.0% w/w or about 3.5% w/w,

In a particular embodiment, the present invention provides a topical composition comprising (i) about 5% to about 20% w/w CBD; and (ii) glycerin. Optionally, the composition further comprises one or more additional components selected from an emollient, an antioxidant, an emulsifier, a preservative and combinations thereof.

In a particular embodiment, the present invention provides a topical composition comprising (i) about 5% to about 20% w/w CBD; and (ii) glycerin, wherein glycerin is present in an amount from about 0.1% w/w to about 10% w/w, or more particularly, from about 1.0% w/w to about 5% w/w, or even more particularly, about 2.5% w/w.

In exemplary embodiments, the present invention provides a topical composition comprising (i) about 5% to about 20% w/w CBD; and (ii) at least one antioxidant. Representative, non-limiting examples of antioxidants include synthetic antioxidants such as BHT (butylalted hydroxytoluene), BHA (butylated hydroxyanisole) and TBHQ (tertiary butyl hydroquinone). Other anti-oxidants suitable for use include acetyl cysteine, ascorbic acid polypeptide, ascorbyl dipalmitate, ascorbyl methylsilanol pectinate, ascorbyl palmitate, ascorbyl stearate, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

In certain embodiments, the at least one antioxidant is present in the composition in an amount from about 0.01% w/w to about 5% w/w, about 0.10% w/w to about 1% w/w, or about 0.10% to about 0.50% w/w. In a particular embodiment, the at least one antioxidant is present in the composition in an amount of about 0.05% w/w, about 0.10% w/w, about 0.20% w/w, about 0.30% w/w, about 0.40% w/w, or about 0.5% w/w.

In a particular embodiment, the present invention provides a topical composition comprising (i) about 5% to about 20% w/w CBD; and (ii) butylalted hydroxytoluene. Optionally, the composition further comprises one or more additional components selected from an emollient, a humectant, an emulsifier, a preservative and combinations thereof.

In a particular embodiment, the present invention provides a topical composition comprising (i) about 5% to about 20% w/w CBD; and (ii) butylalted hydroxytoluene, wherein the butylalted hydroxytoluene is present in an amount from 0.01% w/w to about 5% w/w, more particularly about 0.10% w/w to about 1% w/w, or even more particularly, about 0.20% w/w.

In exemplary embodiments, the present invention provides a topical composition comprising (i) about 5% to about 20% w/w CBD and (ii) at least one emulsifier. Representative, non-limiting examples of emulsifiers include glycol esters, fatty acids, fatty alcohols, fatty acid glycol esters, fatty esters, fatty ethers, esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, derivatives thereof, and mixtures thereof.

In certain embodiments, the at least one emulsifier is present in an amount from about 0.01% w/w to about 5% w/w, about 0.10% w/w to about 1% w/w, or about 0.10% to about 0.50% w/w. In a particular embodiment, the at least one antioxidant is present in the composition in an amount of about 0.05% w/w, about 0.10% w/w, about 0.20% w/w, about 0.30% w/w, about 0.40% w/w, about 0.5% w/w, about 0.6% w/w, about 0.7% w/w, about 0.8% w/w, about 1.0% w/w, about 1.5% w/w, or about 2.0% w/w.

In a particular embodiment, the present invention provides a topical composition comprising (i) about 5% to about 20% w/w CBD; and (ii) polyoxyl stearyl ether.

In a particular embodiment, the present invention provides a topical composition comprising (i) about 5% to about 20% w/w CBD; and (ii) polyoxyl stearyl ether, wherein the polyoxyl stearyl ether is present in an amount from about 0.10% w/w to about 1.0% w/w, or more particularly about 0.40% w/w to about 0.50% w/w, or even more particularly, about 0.47% w/w.

In a particular embodiment, the present invention provides a topical composition comprising (i) about 5% to about 20% w/w CBD; and (ii) PEG 21 stearyl ether.

In a particular embodiment, the present invention provides a topical composition comprising (i) about 5% to about 20% w/w CBD; and (ii) PEG 21 stearyl ether, wherein the PEG 21 stearyl ether is present in an amount from about to about 0.10% w/w to about 10% w/w, or more particularly about 1.0% w/w to about 5% w/w, or even more particularly, about 2.0%.

In a particular embodiment, the present invention provides a topical composition comprising (i) about 5% to about 20% w/w CBD; and (ii) at least two emulsifiers.

In a particular embodiment, the present invention provides a topical composition comprising (i) about 5% w/w to about 20% w/w CBD; and (ii) at least two emulsifiers, wherein the at least two emulsifiers are present in an amount from 0.10% w/w to about 10% w/w, or more particularly about 1.0% w/w to about 5% w/w, or even more particularly, about 2.0% w/w to about 3.0% w/w.

In one embodiment, the present invention provides a topical composition comprising (i) about 5% to about 20% w/w CBD; and (ii) at least two emulsifiers selected wherein the at least two emulsifiers comprise polyoxyl stearyl ether and PEG 21 stearyl ether. Optionally, the composition further comprises one or more additional components selected from an emollient, a humectant, an antioxidant, a preservative and combinations thereof.

In exemplary embodiments, the present invention provides a topical composition comprising (i) about 5% to about 20% w/w gabapentin; and (ii) at least one preservative to inhibit the growth of pathogens over an extended period. Representative, non-limiting examples of suitable preservatives include sorbic acid, benzoic acid, methyl-paraben, propyl-paraben, methyl chloroisothiazolinone, metholisothiazolinone, diazolidinyl urea, chlorobutanol, triclosan, benzethonium chloride, p-hydroxybenzoate, chlorhexidine, digluconate, hexadecyltrimethyl ammonium bromide, alcohols, benzalkonium chloride, boric acid, bronopol, butylparaben, butylene calcium acetate, calcium chloride, calcium lactate, carbon dioxide, cationic, and bentonite, cetrimide, cetylpyridinium chloride, chlorhexidine, chlorobutanol, chlorocresol, chloroxylenol, citric acid monohydrate, cresol, dimethyl ether, ethylparaben, glycerin, hexetidine, imidurea, isopropyl alcohol, lactic acid, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium sorbate, propionic acid, propyl gallate, propylene glycol, sodium acetate, sodium benzoate, sodium borate, sodium lactate, sodium sulfite, sodium propionate, sodium metabisulfite, xylitol, sulphur dioxide, carbon dioxide, and any combination thereof.

Preservatives, when used, are typically present in an amount from about 0.01 to 6 weight percent. In exemplary embodiments, the at least one preservative is present in an amount from about 0.01% w/w to about 5% w/w, about 0.10% w/w to about 5% w/w, about 0.50% w/w to about 5% w/w, or about 1.0% w/w to about 3.0% w/w.

In a particular embodiment, the present invention provides a topical composition comprising (i) about 5% to about 20% w/w CBD; and (ii) at least one preservative selected from propylene glycol, methylparaben and combinations thereof.

In a particular embodiment, the present invention provides a topical composition comprising (i) about 5% to about 20% w/w CBD; and (ii) at least one preservative selected from propylene glycol, methylparaben and combinations thereof, wherein the at least one preservative is present in an amount from about 1.0% w/w to about 5% w/w, or more particularly about 3% w/w to about 4% w/w.

Water may be present in the composition present invention. In exemplary embodiments, water is present in an amount of about 40% to about 99% by weight of the composition, or any range and/or individual value therein, such as, but not limited to, about 55% to about 95% or about 60% to about 70% by weight of the first composition. In certain embodiments, water is present in a first composition of the present invention in an amount of about 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, or 79% by weight of the first composition or any range and/or individual value therein.

The compositions of the present invention optionally include one or more the composition may also include one or more penetration enhancers. Representative, non-limiting penetration enhancers include C₅-C₄₄ fatty alcohols, preferably C₅-C₂₀ fatty alcohols. These fatty alcohols belong to the group of long chain saturated fatty alcohols, unsaturated chain fatty alcohol, branched chain alcohol or combinations thereof.

The compositions of the present invention optionally include one or more stiffening agents. Suitable fragrances and colors may be used in the compositions.

In one embodiment, the present invention is the topical composition disclosed in Example 5.

The composition may be presented in unit dose forms containing a predetermined amount of active ingredient per unit dose. In exemplary embodiments, the unit dosage form is one gram of cream. In alternate embodiments, the cannabidiol may be substituted with another non-psychoactive cannabinoid.

### Combination Therapies

The topical composition can be administered alone or in combination with one or more additional pharmaceutically active agents or prophylactic agents. The one or more additional pharmaceutically active agents or prophylactic agents may include, for example, pain-reducing agents, analgesics, anesthetics, anti-inflammatory agents, anti-seizure agents, antidepressants, anticancer agents, antibiotics, antimicrobial agents, antibacterial agents, antifungal agents, antiviral agents, antiparasitic agents, or anthelmintic agents.

In exemplary embodiments, the topical composition of the present invention may be administered in combination with one or more immunosuppressive agents, anti-inflammatory agents, and/or other agents used in the treatment of pain. These may be formulated in the pharmaceutically acceptable carrier for topical or local administration or administered in a separate formulation, with the other therapy being administered locally (by subcutaneous or intramuscular injection), topically or systemically, to the patient.

Immunosuppressants suitable for use in the methods and compositions of this disclosure include those known in the art. Non-limiting examples include aminopterin, azathioprine, cyclosporin A, D-penicillamine, gold salts, hydroxychloroquine, leflunomide, methotrexate, minocycline, rapamycin, sulfasalazine, tacrolimus (FK506), and pharmaceutically acceptable salts thereof. Other immunosuppressants include, for example, anti-TNF antibodies, such as adalimumab, certolizumab pegol, etanercept, and infliximab. Others include interleukin-1 blockers, such as anakinra. Others include anti-B cell (CD20) antibodies, such as rituximab. Others include T cell activation blockers, such as abatacept.

Anti-inflammatory drugs suitable for use in the methods and compositions of this disclosure include those known in the art. Examples include glucocorticoids and NSAIDs. Examples of glucocorticoids include aldosterone, beclometasone, betamethasone, cortisone, deoxycorticosterone, dexamethasone, fludrocortisones, hydrocortisone, methylprednisolone, prednisolone, prednisone, triamcinolone, and pharmaceutically acceptable salts thereof.

Non-limiting examples of NSAID include salicylates (e.g., aspirin, amoxiprin, benorilate, choline magnesium salicylate, diflunisal, faislamine, methyl salicylate, magnesium salicylate, salicyl salicylate, and pharmaceutically acceptable salts thereof), arylalkanoic acids (e.g., diclofenac, aceclofenac, acemetacin, bromfenac, etodolac, indometacin, nabumetone, sulindac, tolmetin, and pharmaceutically acceptable salts thereof), arylpropionic acids (e.g., ibuprofen, carprofen, fenbufen, fenoprofen, flurbiprofen, ketoprofen, ketorolac, loxoprofen, naproxen, oxaprozin, tiaprofenic acid, suprofen, and pharmaceutically acceptable salts thereof), arylanthranilic acids (e.g., meclofenamic acid, mefenamic acid, and pharmaceutically acceptable salts thereof), pyrazolidine derivatives (e.g., azapropazone, metamizole, oxyphenbutazone, phenylbutazone, sulfinprazone, and pharmaceutically acceptable salts thereof), oxicams (e.g., lornoxicam, meloxicam, piroxicam, tenoxicam, and pharmaceutically acceptable salts thereof), COX-2 inhibitors (e.g., celecoxib, etoricoxib, lumiracoxib, parecoxib, rofecoxib, valdecoxib, and pharmaceutically acceptable salts thereof), and sulphonanilides (e.g., nimesulide and pharmaceutically acceptable salts thereof).

Other agents used in the treatment of pain (including but not limited to neuropathic and inflammatory pain) include, but are not limited to, agents such as pregabalin, lidocaine, duloxetine, gabapentin, carbamazepine, capsaicin, and other serotonin/norepinephrine/dopamine reuptake inhibitors, and opiates (such as oxycontin, morphine, and codeine).

For combination treatment, the one or more additional pharmaceutically active agents or prophylactic agents may be administered separately or in conjunction with the topical composition. In addition, the administration of the one or more additional pharmaceutically active agents or prophylactic agents may be prior to, concurrent to, or subsequent to the administration of the topical composition.

When co-administered with one or more additional pharmaceutically active agents or prophylactic agents, an “effective amount” of the one or more additional pharmaceutically active agents or prophylactic agents will depend on the type of drug used. Suitable dosages are known for approved agents and can be adjusted by the skilled artisan according to the condition of the subject, the type of condition(s) being treated and the amount of a compound described herein being used.

In certain embodiments, the topical composition may be administered in combination with one or more additional therapies selected from the group consisting of iontophoresis, transcutaneous electrical nerve stimulation (TENS), muscle stimulation, diathermy, radiation therapy, infrared, ultraviolet, cold laser or combinations thereof.

### Methods of Treatment

The topical compositions disclosed herein can be used to treat pain, such as neuropathic pain, in a subject. In certain embodiments, the topical compositions can be used to prevent the development of pain, such as neuropathic pain, in a subject. In certain embodiments of the invention, the human subject is in need of treatment by the methods of the invention, and is selected for treatment based on this need. A subject in need of treatment is art-recognized, and includes subjects that have been identified as having pain, has a symptom of such a disease or disorder, or is at risk of such a disease or disorder, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

The composition may be applied locally, for example, topically, to a suitable location. In exemplary embodiments, the compositions may be applied to a suitable area of the skin. In exemplary embodiments, the composition is applied topically to a site at or adjacent to a painful region. The composition may be administered by a medical professional or by the subject.

The composition may be applied by any practical, medically acceptable means. For example, application may be made using fingers, swabs, droppers, squeeze tubes or bottles, compresses, patches, osmotic pumps, aerosols, means for injection, or the like.

Optionally, the treatment location (e.g., skin) may be treated prior to administration of the composition.

Optionally, the application site can be covered with a dressing after the composition has been administered. Suitable dressings may include coverings such as a bandage, which may be porous or non-porous and various inert coverings, e.g., a plastic film wrap or other non-absorbent film. Suitable dressing also encompasses non-woven or woven coverings, particularly elastomeric coverings, which allow for heat and vapor transport. These dressings allow for cooling of the pain site, which provides for greater comfort. In another embodiment, a composition of the invention can be incorporated into a dressing, which dressing is then applied to the skin or painful area.

Administration can be used as a chronic or acute therapy for pain. Pain is defined by the International Association for the Study of Pain (IASP) as “an unpleasant sensory and emotional experience associated with actual or potential tissue damage, or described in terms of such damage”. Pain treatable by this invention is not restricted to any particular cause, disease, or disorder. In exemplary embodiments, administration is used to treat or prevent chronic pain, and more particularly, neuropathic pain, in a subject. In exemplary embodiments, administration is used to treat pain associated with post-surgery or pain due to injury. In addition, compositions and methods of the invention can be used to prevent any acute injury from developing into a chronic pain.

The methods of this invention are useful, for example, for the treatment, reduction, lessening, or prevention of various forms of pain. In exemplary embodiments, the compositions of the present invention are suitable for use in prevention of pain selected from neuropathic, inflammatory, nociceptive, or functional pain.

Exemplary conditions that may be associated with pain include, for example, soft tissue, joint, bone inflammation and/or damage (e.g., acute trauma, osteoarthritis, or rheumatoid arthritis), myofascial pain syndromes (fibromylagia), headaches (including cluster headache, migraine, and tension type headache), myocardial infarction, angina, ischemic cardiovascular disease, post-stroke pain, sickle cell anemia, peripheral vascular occlusive disease, cancer, inflammatory conditions of the skin or joints, diabetic neuropathy, and acute tissue damage from surgery or traumatic injury (e.g., burns, lacerations, or fractures). The present invention is also useful for the treatment, reduction, or prevention of musculo-skeletal pain (after trauma, infections, and exercise), neuropathic pain caused by spinal cord injury, tumors, compression, inflammation, dental pain, episiotomy pain, deep and visceral pain (e.g., heart pain, bladder pain, or pelvic organ pain), muscle pain, eye pain, orofacial pain (e.g., odontalgia, trigeminal neuralgia, glossopharyngeal neuralgia), abdominal pain, gynecological pain (e.g., dysmenorrhea and labor pain), pain associated with nerve and root damage due to trauma, compression, inflammation, toxic chemicals, metabolic disorders, hereditary conditions, infections, vasculitis and autoimmune diseases, central nervous system pain, such as pain due to spinal cord or brain stem damage, cerebrovascular accidents, tumors, infections, demyelinating diseases including multiple sclerosis, low back pain, sciatica, and post-operative pain.

Neuropathic pain generally involves abnormalities in the nerve itself, such as degeneration of the axon or sheath. It is often described as a burning, or shooting type of pain, or tingling or itching pain and may be unrelenting in its intensity and even more debilitating than the initial injury or the disease process that induced it. Neuropathic pain may be distinguished on the basis of etiology and/or location.

In exemplary embodiments, the etiology of the neuropathic pain is post herpetic neuralgia, idiopathic peripheral neuropathy, diabetic neuropathy, chronic radiculopathy, drug or chemotherapy induced neuropathy, chronic regional pain syndrome, trauma, surgery, herniation of an intervertebral disk, spinal cord injury, diabetes, infection with herpes zoster (shingles), HIV/AIDS, traumatic injury, trigeminal neuralgia, erythromelalgia, late-stage cancer, amputation (such as mastectomy), carpal tunnel syndrome, chronic alcohol use or exposure to radiation.

In exemplary embodiments, the method may be used for treatment of pain produced by mixed nociceptive and/or neuropathic mixed etiologies (e.g., cancer, osteoarthritis, fibromyalgia and low back pain), inflammatory hyperalgesia, interstitial cystitis, dermatitis, pruritis, itch, psoriasis, warts, and headaches.

In some embodiments, the composition disclosed herein may be applied to the skin of a subject, e.g., at any suitable location. In some embodiments, the suitable location is a site having neuropathic pain. In some embodiments, the suitable location is near a site having neuropathic pain (e.g., within about 1 cm to about 10 cm, about 10 cm to about 30 cm, about 20 cm to about 50 cm). In some embodiments, the suitable location is an area that covers the site of neuropathic pain and is, for example, about 1 cm, about 2 cm, about 3 cm, about 4 cm, about 5 cm, about 6 cm, about 7 cm, about 8 cm, about 9 cm, about 10 cm, about 15 cm, about 20 cm, about 25 cm, about 30 cm, about 40 cm or about 50 cm in diameter. The composition may be applied to the skin using any suitable method. For example, the composition may be rubbed on, poured on, applied with an applicator (e.g., a gauze pad, a swab, a bandage, etc.), or the like.

The composition is applied such that the dosage is sufficient to provide an effective dose in the painful area or immediately adjacent areas, to ameliorate or eliminate pain and other unpleasant sensations. The dosage will depend on factors such as the age, weight and sex of the patient, condition being treated, severity of the patient's symptoms and response pattern of the patient. It is contemplated that the dosage of the topic composition is administered in unit dosage form and that one skilled in the art would adjust the unit dosage form accordingly to reflect the relative level of activity.

In exemplary embodiments, the dose is between about 1 gram and about 10 grams, between about 1 gram and about 5 grams, or between about 3 grams and about 5 grams of the topical composition.

In exemplary embodiments, the dose is about 1 gram, about 2 grams, about 3 grams, about 4 grams or about 5 grams.

The composition may be provided on a regular schedule, i.e., on a daily, weekly, monthly, or yearly basis or on an irregular schedule with varying administration days, weeks, months, etc. The dose to be administered may vary. In one embodiment, the first dose is higher than the therapeutically effective amount for one or more of the subsequent doses. In another embodiment, the first dose is lower than the one or more of the subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every 2 weeks, about every 3 weeks, about every month, about every 2 months, about every 3 months and about every 6 months. The number and frequency of dosages corresponding to a completed course of therapy will be determined according to the judgment of a health-care practitioner.

In exemplary embodiments, the total daily dose of the topical composition is between about 1 gram and about 10 grams, between about 1 gram and about 5 grams, or between about 3 grams and about 5 grams.

In exemplary embodiments, the total daily dose of the composition is about 1 gram, about 2 grams, about 3 grams, about 4 grams or about 5 grams.

In exemplary embodiments, the topical composition is administered from one to about four times in a 24 hour period, more particularly, one, two, three or four times in a 24 hours period, until the pain or other symptoms have subsided.

In exemplary embodiments, 1 gram of the topical composition is administered every six hours.

In exemplary embodiments, up to 10 grams of CBD is applied each day. For example, between about 8 and about 10 grams, between about 6 and about 8 grams, between about 4 and about 6 grams, between about 2 grams and about 4 grams of CBD. In exemplary embodiments, about 2, about 3, about 4, about 5, about 6, about 7, about 8, about 9 or about 10 grams of CBD is applied each day.

Improvement is present within the context of the present invention if there is a measurable difference between the performances of subjects treated using the methods of the invention as compared to members of a placebo group, historical control, or between subsequent tests given to the same subject. In exemplary embodiments, statistically significant relief is observed based on one or more primary or secondary clinical endpoints compared to a placebo.

In exemplary embodiments, the effect on the subject is assessed by a suitable pain measurement technique or instrument. The pain measurement instrument may be unidimensional or multidimensional.

In exemplary embodiments, the pain experienced by the subject prior to, during and/or after treatment is assessed using the Visual Analogue Scale. A Visual Analog Scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain or itch by choosing the spot on the line that best corresponds to the sensation of pain or itch, where one end of the line corresponds to “no pain” or “no itch” (score of 0 cm) and the other end of the line corresponds to “unbearable pain” or “unbearable itch” (score of 10 cm). This procedure provides a simple and rapid approach to obtaining quantitative information about how the patient is experiencing pain or itch. VAS scales and their use are described, e.g., in U.S. Pat. Nos. 6,709,406 and 6,432,937, the relevant disclosures of which are herein incorporated by reference. In exemplary embodiments, treatment results in about 5% to about 75%, about 10% to about 50%, about 30% to about 50%, or about 20% to about 40% pain reduction on the VAS.

In exemplary embodiments, therapeutic effect (e.g. pain) relief is obtained within minutes to hours and lasts for periods of approximately three to six hours to 24 hours. In exemplary embodiments, therapeutic effect (e.g. pain relief) is obtained within 1 to about 3 minutes, about 3 to about 5 minutes, about 5 to about 10 minutes, about 10 to about 20 minutes, about 20 to about 45 minutes, about 30 minutes, about 45 minutes to 1 hour, about 1 hour, about 1 hour to about 90 minutes, about 120 minutes, about 180 minutes or about 2 hours.

In exemplary embodiments, therapeutic effect (e.g. pain relief) is maintained for a period of about 3 to about 6 hours, about 6 to about 9 hours, about 9 to about 12 hours, about 15 to about 20 hours, or about 20 to about 24 hours. In exemplary embodiments, therapeutic effect (e.g. pain relief) is maintained for a period of about 3 hours, about 4 hours, about 5 hours, about 6 hours, about 7 hours, about 8 hours, about 9 hours, about 10 hours or about 12 hours or more.

In exemplary embodiments, about 90% or more of the maximum therapeutic effect is achieved within about 1 minute, about 3 minutes, about 5 minutes, about 10 minutes, about 15 minutes, about 30 minutes, about 45 minutes or about 1 hour.

In exemplary embodiments, the topical composition is administered with food or following the consumption of food.

Other suitable indices suitable for use in measurement of the subject's pain include, without limitation, the Pain Descriptor Scale (PDS), the Verbal Descriptor Scales (VDS), the Numeric Pain Intensity Scale (NPIS), the Neuropathic Pain Scale (NPS), the Neuropathic Pain Symptom Inventory (NPSI), the Present Pain Inventory (PPI), the Geriatric Pain Measure (GPM), the McGill Pain Questionnaire (MPQ), mean pain intensity (Descriptor Differential Scale), numeric pain scale (NPS), global evaluation score (GES), the Short-Form McGill Pain Questionnaire, the Minnesota Multiphasic Personality Inventory, the Pain Profile and Multidimensional Pain Inventory, the Child Heath Questionnaire, and the Child Assessment Questionnaire.

In exemplary embodiments, the use of topical administration in certain embodiments of the present invention has various advantages compared to other forms of administration. In a particular embodiment, the topical composition is more effective compared to the forms of administration. In another particular embodiment, the topical composition is more stable and/or has a longer shelf-life than other forms of administration. In yet another embodiment, the topical composition produces fewer side effects than other forms of administration.

In exemplary embodiments, the method produces pain relief without sedation, without central nervous adverse effects and/or without addiction.

The present invention extends to regimens, kits or packages of the topical composition.

In exemplary embodiments, the topical composition is provided in a kit. The kit may contain packaging or a container with topical composition or the components of the topical composition, with instructions for mixing the components to prepare the composition. Optionally, the kit may contain instructions on dosing. The kit may optionally contain instructions for administering the composition to the subject and/or an insert regarding the pharmaceutically active ingredients. Optionally, the kit may further contain instructions for monitoring the subject's response to treatment. Such kits are readily packaged in a manner suitable for treatment of a desired indication. For example, the kit may also contain instructions for using a delivery device. Other suitable components to include in such kits will be readily apparent to one of skill in the art, taking into consideration the desired indication.

In exemplary embodiments, the topical composition is provided in packaging. The packaging may itself be geared for administration, such as a pump or other apparatus, from which the formulation may be applied to an affected area of the body, such as the lungs, injected into a subject, or even applied to and mixed with the other components of the kit.

### Methods of Manufacture

The topical compositions of the invention are compounded with the pharmaceutically acceptable carrier, then packaged and stored.

In exemplary embodiments, the components of the composition are added in the following order: (i) the at least one emollient; (ii) the at least one antioxidant; (iii) the at least one emulsifier (melted); (iv) water and glycerin; (v) the at least one preservative; (vi) the gabapentin and (vii) the ketamine HCL. In alternative embodiments, the order of addition varies.

A preferred, but non-limiting method for manufacture of the composition comprises (i) melting the at least one emulsifier (e.g., place in an oven set to about 70° C.); (ii) adding the at least one emollient into a container (e.g., a stainless steel container); (iii) mixing the at least one emollient with a mixer and mixing blade while heating (e.g., using a hot plate) to about 60° C.; (iv) adding the at least one antioxidant to the at least one emollient to form a first mixture; (v) continuing to mix the first mixture, while maintaining the temperature at about 60° C.; (vi) adding the melted emulsifier to the first mixture to form a second mixture; (vii) continuing to mix the second mixture while maintaining the temperature at about 60° C.; (ix) mixing sterile water and glycerin in a separate container to provide a third mixture; (x) adding the second mixture to a (heated) Ekato mixer under suitable conditions (e.g., mixing counter-clockwise with an agitator speed of about 150 rpm and a homogenizer speed of about 3000 rpm, while maintaining the temperature at about 60° C.; (xi) adding the third mixture to the second mixture to form a fourth mixture; (xii) mixing the fourth mixture for about 10 minutes while maintaining the temperature at about 60° C.; (xii) mixing the at least one preservative with a mixer and mixing blade until substantially all of the preservative has been dissolved, forming to provide a fifth mixture; (xiii) adding the fifth mixture to the fourth mixture to provide a sixth mixture; (xiv) adding a desired amount of gabapentin to the sixth mixture to form a seventh mixture, while continuing to mix; (xv) adding a desired amount of ketamine HCL to the seventh mixture to form an eighth mixture; and (xvi) mixing the eighth mixture for about 10 minutes, to provide the composition of the present invention. Optionally, the mixture can be discharged into a container.

In exemplary embodiments, the components of the composition are added in the following order: (i) the at least one emollient; (ii) the at least one antioxidant; (iii) the at least one emulsifier (melted); (iv) water and glycerin; (v) the at least one preservative; and (vi) the CBD. In alternative embodiments, the order of addition varies.

The present invention and its many attendant advantages will be understood from the foregoing description and it will be apparent that various changes in form, construction and arrangement of the parts thereof may be made without departing from the spirit and scope of the invention or sacrificing all of its material advantages, the form hereinbefore described are merely exemplary embodiments thereof.

### EXAMPLES

### Example 1: Formulation

In formulating a batch of a representative composition of the present invention, a particularly effective product resulted when the following components were utilized within the approximate ranges shown in Table 1, below:

TABLE 1

| | Chemical | | |
|---|---|---|---|
| Ingredient | Description | Manufacturer | % w/w |
| Crodamol IPM | Isopropyl | Croda | 10.00% |
| | Myristate | | |
| Crodacol C95 | Cetyl Alcohol | Croda | 5.00% |
| Crodacol S95 | Stearyl Alcohol | Croda | 5.00% |
| Glycerine | Glycerine | Specrum | 2.50% |
| BHT | Butylated | Sigma- | 0.20% |
| | Hydroxytoluene | Aldrich | |
| Brij S2 | Polyoxyl Stearyl | Croda | 0.47% |
| | Ether | | |
| Brij S721 | PEG21 Stearyl | Croda | 2.03% |
| | Ether | | |
| Sterile Water | Water | HyClone | 61.60% |
| Propylene Glycol | Propylene Glycol | Specrum | 3.00% |
| Methylparaben | Methylparaben | Univar | 0.20% |
| Gabapentin | Gabapentin | Letco | 5.00% |
| Ketamine HCl | Ketamine HCl | Letco | 5.00% |
| Total | Emerson | Emerson | 100.00% |

### Example 2: Manufacture

In formulating a batch of a representative composition of the present invention, a particularly effective product resulted when the following manufacturing protocol was utilized:
1) Place the Brij S2 and Brij S721 in an oven set to 70° C.
2) Dispense and add the Crodamol IPM into a stainless steel container.
3) Dispense both the Crodacol C95 and Crodacol S95 and add them to the stainless steel container containing the Crodamol IPM.
4) Mix the solution with a mixer and mixing blade while heating on a hot plate to approximately 60° C.
5) Dispense and add the BHT to the solution while continuing to mix and maintain mixing temperature of approximately 60° C.
6) Once the Brij S2 has melted, dispense and add it to the solution while continuing to mix and maintain mixing temperature of approximately 60° C.
7) Once the Brij S721 has melted, dispense and add it to the solution while continuing to mix and maintain mixing temperature of approximately 60° C.
8) Dispense the Sterile Water.
9) Dispense and add the Glycerine to the Sterile Water while mixing with a mixer and mixing blade.
10) Heat the Ekato mixer to approximately 60° C.
11) Add the oil phase to the Ekato mixer while mixing counter-clockwise with an agitator speed of 150 RPM and homogenizer speed of 3000 rpm. Maintain temperature of approximately 60° C.
12) Slowly add the Treated Water and Glycerine to the Ekato mixer while mixing
13) Once all the water has been added, mix for 10 mins and maintain mixing temperature of approximately 60° C.
14) Dispense the Propylene Glycol into a container and begin to stir with a mixer and mixing blade.
15) Dispense and add the Methylparaben to the Propylene Glycol while mixing. Mix until all the Methylparaben has been dissolved.
16) Add the Propylene Glycol/Methylparaben solution to the Ekato mixer.
17) Dispense and add the Gabapentin to the emulsion in the Ekato mixer while continuing to mix
18) Dispense and add the Ketamine HCl to the emulsion while continuing to mix.
19) Mix for 10 mins.
20) Discharge the emulsion from the Ekato mixer into a container.

### Example 3: Stability

Stability testing was conducted, with results at two weeks shown in Table II, below:

TABLE II

| | | | | | | | Total |
|---|---|---|---|---|---|---|---|
| Sample | Name | Assay | Name | Assay | Name | Assay | Impurities |
| t = 0 | Gabapentin | 111.9 | Ketamine HCl | 108.8 | Methylparaben | 111.1 | 0.0 |
| | Gabapentin | 110.7 | Ketamine HCl | 103.9 | Methylparaben | 112.9 | 0.0 |
| | avg | 111.3 | avg | 106.3 | avg | 112.0 | 0.0 |
| 2 wk, | Gabapentin | 105.4 | Ketamine HCl | 105.9 | Methylparaben | 108.2 | 0.0 |
| 25/60 | Gabapentin | 107.8 | Ketamine HCl | 105.7 | Methylparaben | 104.1 | 0.0 |
| | avg | 106.6 | avg | 105.8 | avg | 106.1 | 0.0 |
| 2 wk, | Gabapentin | 106.1 | Ketamine HCl | 105.2 | Methylparaben | 109.6 | 0.3* |
| 40/75 | Gabapentin | 105.5 | Ketamine HCl | 105.2 | Methylparaben | 106.7 | 0.2* |
| | avg | 105.8 | avg | 105.2 | avg | 108.1 | 0.3* |

*Single impurity RRT 1.54)

### Example 4: In Vitro Skin Absorption Testing

In vitro skin absorption of gabapentin and ketamine formulations is tested using pig skin model.

Pig skin is obtained from the abdomen of female weanling Yorkshire pigs (NCSU Unit Two, Lake Wheeler Rd, Raleigh, N.C.). Skin is carefully dermatomed to a thickness of 500 um using a Padgett dermatome. Circular skin sections is placed epidermal side up into each Teflon Bronaugh flow-through diffusion cell to provide a dosing area of 0.64 cm². The epidermal side is dosed with one of three formulations containing gabapentin, ketamine, or gabapentin+ketamine with seven (7) skin replicates per formulation. The dermal side in each cell is bathed with receptor fluid (Krebs-Ringer bicarbonate buffer containing albumin and glucose with pH=7.4) at a set flow rate (4 ml/hr) by a multichannel peristaltic cassette pump. The receptor fluid mimics in vivo plasma and facilitates absorption of lipid soluble solutes. Perfusate samples are collected at 0, 1.0, 2.0, 4.0, and 6.0 hrs. The I 05 perfusate samples (7 replicates×3 formulations×5 time points) are analyzed by for the two drugs.

### Example 5: Formulation

In formulating a batch of a representative composition of the present invention, a particularly effective product results when the following components were utilized within the approximate ranges shown in Table 1, below:

TABLE 1

| | Chemical | | |
|---|---|---|---|
| Ingredient | Description | Manufacturer | % w/w |
| Crodamol IPM | Isopropyl | Croda | 10.00% |
| | Myristate | | |
| Crodacol C95 | Cetyl Alcohol | Croda | 5.00% |
| Crodacol S95 | Stearyl Alcohol | Croda | 5.00% |
| Glycerine | Glycerine | Spectrum | 2.50% |
| BHT | Butylated | Sigma- | 0.20% |
| | Hydroxytoluene | Aldrich | |
| Brij S2 | Polyoxyl Stearyl | Croda | 0.47% |
| | Ether | | |
| Brij S721 | PEG21 Stearyl | Croda | 2.03% |
| | Ether | | |
| Sterile Water | Water | HyClone | 61.60% |
| Propylene Glycol | Propylene Glycol | Spectrum | 3.00% |
| Methylparaben | Methylparaben | Univar | 0.20% |
| CBD | Cannabidiol | | 10.00% |
| Total | Emerson | Emerson | 100.00% |

1. A topical pain-relief composition comprising (i) about 5% gabapentin; (ii) about 5% ketamine; and (iii) a pharmaceutically acceptable carrier.

2. The composition of claim 1, wherein the topical composition further comprises one or more additional components selected from at least one emollient, at least one humectant, at least one antioxidant, at least one emulsifier, at least one preservative and combinations thereof.

3. A topical pain-relief pain composition comprising (i) about 5% gabapentin; (ii) about 5% ketamine; (iii) at least one emollient selected from isopropyl myristate, cetyl alcohol, stearyl alcohol or combinations thereof; (iv) glycerine; (v) butylated hydroxytoluen; (vi) at least one emulsifier selected from polyoxyl stearyl ether, PEG-21 stearyl ether and combinations thereof; (vii) at least one preservative selected from propylene glycol, methylparaben and combinations thereof; and (viii) water.

4. A method of treating pain in a subject in need thereof, comprising topically administering to the subject a composition comprising (i) about 5% gabapentin; (ii) about 5% ketamine; and (iii) a pharmaceutically acceptable carrier, thereby treating pain in the subject.

5. The method of claim 4, wherein the topical composition further comprises one or more additional components selected from at least one emollient, at least one humectant, at least one antioxidant, at least one emulsifier, at least one preservative and combinations thereof.

6. The method of claim 4, wherein the pain is chronic pain.

7. The method of claim 4, wherein the pain is neuropathic pain.

8. The method of claim 7, wherein the neuropathic pain is selected from post herpetic neuralgia, idiopathic peripheral neuropathy, diabetic neuropathy, chronic radiculopathy, drug or chemotherapy induced neuropathy or chronic regional pain syndrome.

9. The method of claim 4, wherein the total daily dose is between about 1 and about 5 grams of the topical composition.

10. The method of claim 9, wherein the total daily dose is between about 1 and about 3 grams of the topical composition.

11. The method of claim 10, wherein the topical daily dose is about 1 gram of the topical composition.

12. The topical composition of claim 10, wherein the total daily dose is about 2 grams of the topical composition.

13. The method of claim 10, wherein the topical daily dose is about 3 grams of the topical composition.

14. The method of claim 9, wherein the total daily dose is between about 4 grams and about 5 grams of the topical composition.

15. The method of claim 14, wherein the topical daily dose is about 4 grams of the topical composition.

16. The topical composition of claim 14, wherein the total daily dose is about 5 grams of the topical composition.

17. The method of claim 4, wherein the topical composition is administered once a day.

18. The method of claim 4, wherein the topical composition is administered at least twice a day.

19. The topical composition of claim 4, wherein about 1 gram of the composition is administered every six hours.

20. The method of claim 4, wherein the pain is treated without sedation, central nervous adverse effects, addition or a combination thereof.

21. A topical pain-relief composition comprising (i) about 5% to about 20% cannabidiol (CBD); and (ii) a pharmaceutically acceptable carrier.

22. The composition of claim 21, comprising about 5% to about 10% cannabidiol.

23. The composition of claim 21, further comprising one or more additional components selected from at least one emollient, at least one humectant, at least one antioxidant, at least one emulsifier, at least one preservative and combinations thereof.

24. A method of treating pain in a subject in need thereof, comprising topically administering to the subject a composition comprising (i) about 5% to about 20% cannabidiol; and (ii) a pharmaceutically acceptable carrier, thereby treating pain in the subject.

25. The method of claim 24, wherein the total daily dose of cannabidiol is about 10 grams or less.

26. The method claim 24, wherein the topical composition is administered once a day.

27. The method of claim 24, wherein the topical composition is administered at least twice a day.

28. The method of claim 37, wherein the pain is treated without sedation, central nervous adverse effects, addition or a combination thereof.
